(19) United States
(12) Patent Application Publication
(10) Pub. No.: US 2009/0318399 A1
(43) Pub. Date: Dec. 24, 2009

(54) PHARMACEUTICAL COMPOSITION AND EXTRACT OF PORIA FOR TREATING A DISEASE INDUCED FROM IMMUNE DISORDER

(75) Inventors: Hang-Ching Lin, Taipei (TW); Wen-Mein Wu, Taipei (TW)

Correspondence Address: OCCHIUTI ROHLICEK & TSAO, LLP, 10 FAWCETT STREET, CAMBRIDGE, MA, 02138, US

(73) Assignee: Sinphar Pharmaceutical Co., Ltd., I Lan (TW)

(21) Appl. No.: 12/473,110

(22) Filed: May 27, 2009

(30) Foreign Application Priority Data
Jun. 20, 2008 (TW) 97123189

(51) Int. Cl. A61K 31/56

(52) U.S. Cl. 514/180; 514/169; 514/182

(57) ABSTRACT

The present invention discloses a novel use of a lanostane having the following formula (I) in treating a disease induced from immune disorder:

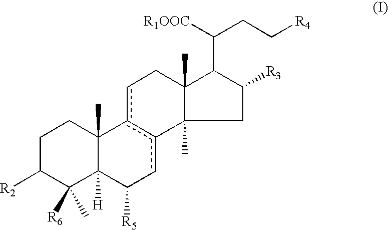

wherein R₁ is either H or CH₃; R₂ is OCOCH₃, ═O or OH; R₃ is H or OH; R₄ is —C(═CH₂)—C(CH₃)₂Rₐ, in which Rₐ is H or OH, or —CH═C(CH₃)—R_{b}, in which R_{b} is CH₃ or CH₂OH; R₅ is H or OH; and R₆ is CH₃ or CH₂OH.

### FIELD OF THE INVENTION

The present invention relates to a novel use of lanostane compounds in treating a disease induced from immune disorder, such as allergies.

### DESCRIPTION OF PRIOR ART

Immunoglobulin E (IgE) is one class of immunoglobulin (or “antibody”) molecule. IgE is present in human serum in lower concentrations than the other immunoglobulins: IgG, IgM, IgA, and IgD. IgE is thought to have a role in protection against parasites, but has never been definitively established as playing a necessary, or even a beneficial role, at least in developed countries, where parasite infections are not a significant problem. IgE is well known as the mediator of immediate-type hypersensitivity allergic reactions, including allergic rhinitis (“hay fever”), asthma, urticaria and food and drug allergies.

In IgE-mediated allergic reactions, IgE, after it is secreted by B cells, binds through its Fc portion to the FcεRI receptors, which are present on the surface of basophils, mast cells and Langerhans cells. If the IgE bound to the surface of these cells now contacts and binds an allergen, this causes a cross-linking of the bound IgE molecules and hence the underlying receptors, and triggers the release of pharmacologic mediators, such as histamine, serotonin, leukotrienes and the slow reacting substance of anaphylaxis. These mediators cause the pathologic manifestations of allergic reactions.

Some patients with a history of some or all of the IgE-mediated allergic conditions also suffer from a painful skin condition called atopic dermatitis.

Among the allergic disease taking asthma as an example the immune reaction time can be divided into an immediate asthmatic response and a late asthmatic response. The immediate asthmatic response is a response caused by the inflammatory mediators released from the mast cells, which occurs within 15 to 30 minutes following the contact of the allergen. The allergen identifies and binds to IgE bound to the mast cells, when the patient is exposed to the same allergen again, activating the mast cell, and granules in the cells undergoing degranulation, so that more inflammatory substances are released, including histamine, leukotrienes, cytokines such as IL-2, IL-4, IL-5 and GM-CSF, and chemoattractant factors, etc., and thus the permeability of the vessels increases and the smooth muscle of the airway contracts. These inflammatory substances, cytokines and chemoattractant factors not only affect the immediate asthmatic response, but the late inflammatory response.

The response caused by eosinophils and neutrophils pertains to the late response. 4 to 6 Hours after the immediate response the cytokines and chemoattractant factors released from the mast cells will attract the inflammatory cells such as eosinophils and neutrophils, resulting in infiltration. Further, Cytokines such as Eotaxin secreted from epithelial cells, endothelial cells and fibroblast cells are the major portion of the chemoattractant factors, which also cause eosinophils or Th2 immune cells moving to the inflammatory sites at the bronchi in the lungs. Many inflammatory proteins such as MBP (Major Basic Protein), ECP (Eosinophil Cationic Protein), Eosinophil Derived Neurotoxin, EPO (Eosinophil Peroxidase), and leukotrienes, etc. will be secreted from eosinophils, when they are activated. These inflammatory proteins irritate the smooth muscle of the airway to contract, increasing the permeability of the vessels, and causing the airway edema, so that the epithelial tissues of the airway are hurt directly, and thus the epithelial cells lose their integrity. The airway secretes too much mucus, which not only blocks or narrows the airway, but causes infiltration of neutrophils and degranulation of the mast cells, thereby increasing infiltration of eosinophils. As a result, the degree of asthma is more severe.

US 2004/0229852 A1 discloses a pharmaceutical composition useful in enhancing immunity of human body. The composition contains potent components of lanostane compounds. A method is devised to obtain an extract from metabolite, sclerotium, or fermentation product of Poria cocos (Schw) Wolf. The extract contains 5-60% of the lanostane compounds by weight of the extract. The extract is devoid of secolanostane capable of inhibiting immunity development.

A substance or method capable for treating an allergy is an important issue not only to researchers but also to ordinary people alike. It has not been reported so far that a lanostane compound is potent in treating an allergy.

### SUMMARY OF THE INVENTION

A primary objective of the present invention is to provide a novel use of lanostane compounds in treating a disease induced from immune disorder such as allergy.

Another objective of the present invention is to provide a pharmaceutical composition for treating a disease induced from immune disorder (for example allergy) comprising a lanostane compound as a potent component.

Another objective of the present invention is to provide a method for treating a disease induced from immune disorder (for example allergy) by using a lanostane compound.

A pharmaceutical composition capable of treating a disease induced from immune disorder (for example allergy) in a mammal (for example, a human), which comprises an amount effective for treating said disease of a lanostane having the following chemical formula (I) as an active ingredient:

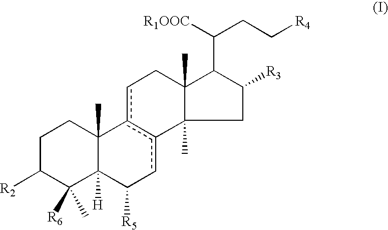

wherein R₁ is either H or CH₃; R₂ is OCOCH₃, ═O or OH; R₃ is H or OH; R₄ is —C(═CH₂)—C(CH₃)₂Rₐ, in which Rₐ is H or OH, or —CH═C(CH₃)—R_{b}, in which R_{b} is CH₃ or CH₂OH; R₅ is H or OH; and R₆ is CH₃ or CH₂OH, or a pharmaceutically acceptable salt thereof.

The present invention also provides a use of the lanostane having the above formula (I) in the fabrication of a medicament for treating a disease induced from immune disorder in a mammal.

Preferably, the allergy is allergic rhinitis, allergic conjunctivitis, allergic asthma, atopic dermatitis, food allergy, atopic eczema, or rheumatoid arthritis. Most preferably, said allergy is allergic asthma.

Preferably, the lanostane having the following chemical formula (I) is

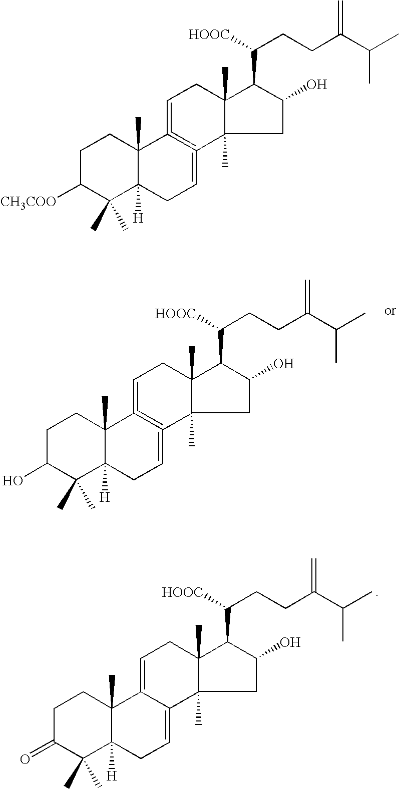

Preferably, the pharmaceutical composition comprises 0.1-60% of the lanostane (I) or a pharmaceutically acceptable salt thereof by weight of the composition.

The pharmaceutical composition for treating an allergy of the present invention in general can be administered to the patient orally in the form of such as a tablet, pill, soft capsule, hard capsule, micro granule, powder or pellet, etc. The pharmaceutical composition of the present invention can be in the form of a liquid, which can be administered orally or non-orally as an aqueous solution. The pharmaceutical composition of the present invention can be dispersed in ethanol, water or the like miscible solvent, and administered non-orally in the form of a spray, lotion, ointment, tincture, emulsion or ampoule.

Preferably, the pharmaceutical composition is orally administered. A further objective of the present invention is using a Poria extract as a source of said lanostane (I). Said Poria extract comprises 1-60% of the lanostane (I) by weight of the extract, and being substantially devoid of secolanostane.

Preferably, said Poria extract is prepared by a method comprising the following steps:

a) extracting metabolites, fermentation products or sclerotium of Poria cocos (Schw) Wolf by water, methanol, ethanol, or a mixed solvent thereof;
b) concentrating the resulting liquid extract from step a);
c) introducing the resulting concentrated substance from step b) into a silica gel column;
d) eluting the silica gel column with an eluent having a low polarity, and collecting the resulting eluate; and
e) concentrating the eluate to form a concentrated eluate.

Preferably, the concentrated eluate from step e) has a chromatographic value, Rf, not less than 0.1 in accordance with a thin layer chromatography, which is developed by a mixed solvent of dichloromethane:methanol=96:4 and is detected by an ultraviolet lamp and iodine vapor.

Preferably, the extraction in step a) is carried out by using 95% ethanol.

Preferably, the extraction in step a) comprises extracting metabolites, fermentation products or sclerotium of Poria cocos (Schw) Wolf by boiling water; adding a base to the resulting extraction aqueous solution until a pH value thereof is 9-11; recovering the basic aqueous solution; adding an acid to the basic aqueous solution until a pH value thereof is 4-6 to form a precipitate; recovering the precipitate; extracting the precipitate with ethanol; and recovering a liquid extract.

Preferably, the concentrated substance resulted from step b) is further extracted with a two-phase solvent containing methanol and n-hexane in a volumetric ratio of 1:1, a methanol layer is separated from the two-phase solvent extraction mixture, and the methanol layer is concentrated to form a concentrate, which is used as a feed to the silica gel column in step c).

Preferably, the low polarity eluent in step d) is a mixed solvent containing dichloromethane and methanol in a volumetric ratio of 96.5:3.5.

Preferably, said Poria extract comprises 5-35% of the lanostane (I).

Preferably, the pharmaceutical composition of the present invention further comprises a diluent, excipient or carrier.

### DETAILED DESCRIPTION OF PREFERRED EMBODIMENTS

In the following experiments conducted herein the mice were induced to become allergic by using ovalbumin (OVA) as an allergen, and the mice were confirmed suffering the induced asthma from the appearance of OVE-specific IgE antibody in the mice. Mice in different groups were fed with a Poria extract or pure lanostane compound during the experimental period, and each mouse was subjected to an airway hyperresponsiveness test after one-month feeding, from which an important index reflecting the severe degree of the asthma of the mouse was determined. The inventors of the present invention also observed different immune cells in bronchoalveolar lavage fluid (BALF) of the mice as to whether they particularly eosinophils and neutrophils were affected. Further, a chemoattractant factor, Eotaxin, was an important observation factor in the present invention for observing whether the secretion of cytokines changed dramatically. The observations of the above-mentioned three pharmacological or inflammatory substances, airway hyperresponsiveness, inflammatory cells and chemoattractant factor (Eotaxin), reveal that the Poria extract and lanostane compounds are an excellent medicine for the prophylaxis and treatment of asthma.

An extract of Poria for enhancing nutrient uptake by mammals (for example, humans) disclosed in the present invention can be prepared by a process similar to that disclosed in US2004/0229852 A1, which includes extracting Poria cocos (Schw) Wolf with the conventional extraction methods to obtain a crude extract, separating the crude extract by chromatography into a low polarity fraction of lanostane (with an eluent of dichloromethane:methanol of 96:4) and a high polarity fraction of secolanostane (with eluents of dichloromethane:methanol of 90:10, and 0:100), wherein the lanostane fraction is detected by a thin layer chromatography having a chromatographic value, Rf, not less than 0.1 in accordance, when it is developed by a mixed solvent of dichloromethane:methanol=96:4; the Rf is less than 0.1 for the secolanostane fraction. Several lanostanes are separated from the lanostane fraction by subjecting the lanostane fraction to silica gel column chromatography eluted, wherein the eluents used are dichloromethane:methanol=97:3 to 95:5.

The following examples are provided for describing the present invention in further details, but should not be used to limit the scope of the present invention.

Percentages and other amounts referred to in this specification are by weight unless indicated otherwise. Percentages are selected from any ranges used to total 100%.

### Example 1

A Poria powder was made of 30 kilograms of the China-grown Poria cocos (Schw) Wolf. The Poria powder was extracted with 120 L 95% alcohol for 24 hours. The mixture was filtered to obtain a filtrate. The residue was extracted and filtered for another three cycles. The filtrates were combined and concentrated to bring about a dried extract in amount of 265.2 grams. The dry extract was undergone a distribution extraction with a two-phase extraction agent (n-hexane:95% methanol=1:1), and the methanol layer was removed therefrom, which is then concentrated to obtain a dry solid in an amount of 246.9 grams. A separation of the dry solid was carried out by means of a silica gel column, which was filled with silica gel 10-40 times of the weight of the dry solid. The silica gel having a diameter of 70-230 mesh was made by Merck Corporation with a code of Silica Gel 60. The column was eluted by the following eluates in sequence: a mixed solvent of dichloromethane: methanol=96:4; a mixed solvent of dichloromethane:methanol=90:10, and pure methanol. The eluates were tested by the thin layer chromatography (TLC), wherein an ultraviolet lamp and iodine vapor were used for detecting, and a mixed solvent of dichloromethane:methane=96:4 was used as a developing liquid. The eluates having similar constituents in the TLC were combined.

The elution carried out with the mixed solvent of dichloromethane:methanol=96:4 resulted in a PCM portion in amount of 78 grams. The PCM shows 6 trace points in the thin layer chromatography. The resulting eluates from the elutions carried out with the eluents of dichloromethane:methanol=90:10 and pure methanol were combined to obtain a PCW portion in amount of 168 grams.

The PCM portion was further separated by means of an eluent of dichloromethane:methanol=96.5:3.5 and the same silica gel column to obtain purified lanostane components of K1 (K1-1 and K1-2), K2 (K2-1 and K2-2), K3, K4, K4a, K4b, K5, K6a and K6b. Further details of the separation steps and identification analysis data can be seen in US2004/0229852 A1.

The aforesaid K1 to K6b compounds have the following structures:

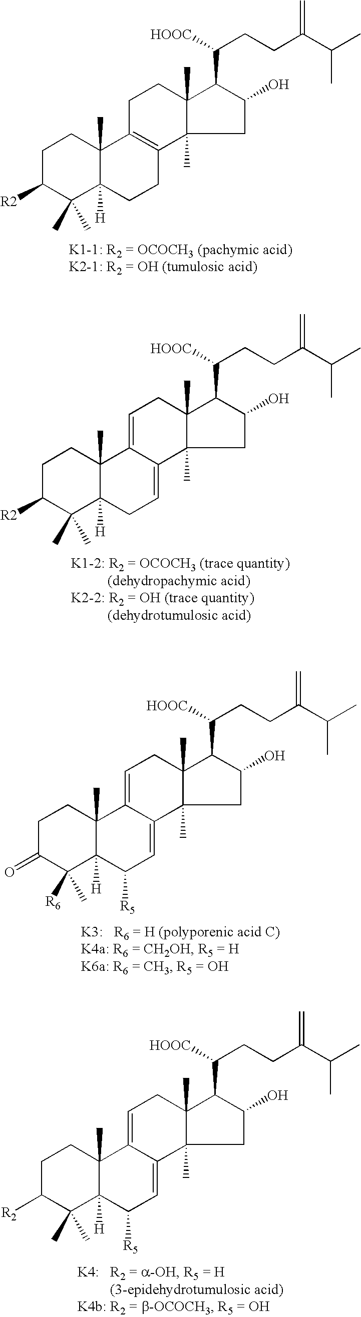

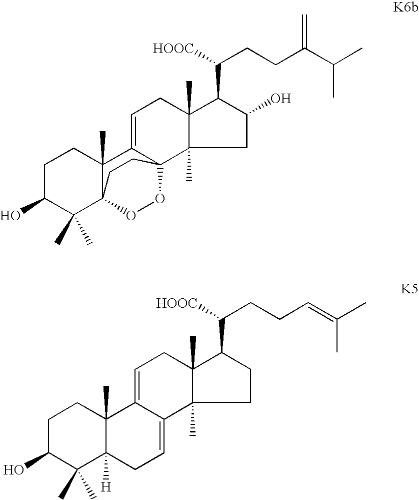

The amounts of the lanostane compounds K1 to K6b separated from the PCM portion are listed in the table below. The PCM portion contains approximately 15 wt % of the lanostane compounds K1 to K6b.

| K1 | K2 | K3 | K4 | K4a | K4b | K5 | K6a | K6b |
|---|---|---|---|---|---|---|---|---|
| 3.0 g | 6.2 g | 1.93 | 0.55 | 66 mg | 86.8 mg | 47.6 mg | 21.4 mg | 90.7 |
| | | g | g | | | | | mg |

### Example 2

100 kg of Poria was boiled with 800 kg of water for 3 hours, then left for cooling to 50° C. and a pH value thereof was adjusted to pH 11 by using a 5N NaOH solution, followed by stirring the resulting solution for 3 hours. A centrifugation machine was used to separate the liquid from the solid, followed by adding another 800 kg of water to the separated solid. The aforesaid procedures were repeated, including adjusting pH value with NaOH to pH 11, stirring, and removing the solid by centrifugation. The two resulting liquids were combined, and then vacuum concentrated to a solution of 100 kg at 50° C., followed by the adjustment of pH value to pH 6.5 by using 3N HCl so as to produce a precipitate. Said precipitate was separated from the solution, subsequently rinsed with 40 L H₂O, and centrifuged in order to recover the precipitate; the precipitate was sprayed dry with 8 L of water, which yielded 380 g of powder. Afterwards, the powder was extracted three times by using 4 L of alcohol, and the extraction solutions were combined and concentrated to result in 238.9 g of alcohol extract. The 238.9 g of alcohol extract was proved containing no secolanostane compound by the TLC analysis in Example 1, and then was subject to HPLC separation, which gave 185.93 mg of K2, 20.34 mg of K3, 15.82 mg of K4, and 4.52 mg of K1 per gram of the extract. In other words, each gram of the extract has approximately 226.07 mg of lanostane compounds.

### Example 3

### Experiments of Treating the Asthmatic Mice with Poria Extract or Pure Lanostane Compounds

In this example the mice were induced to become allergic by using ovalbumin (OVA) as an allergen, and the mice were confirmed suffering the induced asthma from the appearance of OVE-specific IgE antibody in the mice. Mice in different groups were fed with a Poria extract or pure lanostane compound during the experimental period, and each mouse was subjected to an airway hyperresponsiveness test after one-month feeding, from which an important index reflecting the severe degree of the asthma of the mouse was determined. In this example different immune cells in bronchoalveolar lavage fluid (BALF) of the mice were also observed as to whether they particularly eosinophils and neutrophils were affected. Further, a chemoattractant factor, Eotaxin, was an important observation factor in this example for observing whether the secretion of cytokines changed dramatically.

(1) Experimental animal: Inbred BALB/c female mice were maintained by standard laboratory chow ad libitum. Room temperature was maintained at 19-24° C. and relative humidity at 50-70%. Animal experiments were performed according to the Guidelines for the Care and Use of Laboratory Animals, Fu-Jen University, Taiwan. BALB/c mice were divided into 10 groups each of which had 8 mice.
(2) Establish of the experimental asthma model of animals: The immunization protocol was similar and modified according to that described previously (Sy, L. B, et al. Propolis extracts exhibit an immunoregulatory activity is an ova-sensitized airway inflammatory animal model. Int Immunopharm 6:1053-1060. (2006). Briefly, the BALB/c mice were immunized with an intraperitoneal injection of 10 μg/ml and 30 μg/ml OVA (Albumin, chicken egg, A-5503, Sigma) with the 2 mg adjuvant aluminium hydroxide (Al(OH)₃, 77161, Pierce) at 8 and 10 weeks of age, respectively. All mice were exposed to inhalate exposure to 8 ml of 2% OVA aerosols for a period of 20 minutes by placing them in a chamber using an ultrasonic nebulizer (DeVilbiss Pulmo-Aide, 5650D, USA), after receiving the second intraperitoneal injection.
(3) Grouping of the experimental animal: The 10 groups of OVA-sensitized asthmatic mice were separated into a non-treatment asthma group (As); treatment groups with the Poria extract prepared in Example 2 and pure compounds K1, K2 and K3 (1PCE, 1K1, 1K2, 1K3, 2PCE, 2K1, 2K2, and 2K3); and the control drug group (Pred). Each mouse in the treatment group of 1PCE group received daily 0.0372 mg of the ethanol extract PCE prepared in Example 2 (1PCE). 2PCE means mice were fed with two times of the dosage of PCE (0.0744 mg) of the 1PCE group. Each mouse in the treatment groups of 1K1, 1K2 and 1K3 received daily 0.0087 mg of K1, K2 and K3, respectively. 2K1, 2K2, and 2K3 mean mice were fed with two times of the dosage of K1, K2 and K3 (0.0174 mg) in comparison with the 1K1, 1K2 and 1K3 groups. Each mouse of the Pred group had been administrated with 0.1 mg of Prednisolone per day for 5 continuous days before the mouse was sacrificed. Above PCE, K1, K2, K3 and Prednisolone compounds were dissolved by 95% ethanol firstly, and finally in Phosphate Buffer Solution (PBS). Each mouse was administered 0.4 mL of total volume each time by gavage.

### (4) Airway Hyperresponsiveness Test:

An airway hyperresponsiveness (AHR) value increases proportionally with the severe degree of asthma, and thus a decrease of AHR value is an important index for determining whether asthma is being released. Each mouse in all groups was subjected to the AHR test next day following the 29^{th} day of administering in the treatment groups on which the last inhalate exposure was conducted. The AHR was tested with Buxco system (Biosystem XA; Buxco Electronics Inc. Sharon, Conn., USA), wherein the mouse was placed in a chamber, and aerosol of PBS or methacholine of different concentrations (25 mg/ml, and 50 mg/ml) was introduced into the chamber by using an ultrasonic nebulizer. Three minutes after the introduction an average value of AHR per minute was recorded. The contraction of airway induced by methacholine was more significant when the concentration of methacholine was increased. The value of Penh (pause of enhance) was calculated by collecting data derived from transducer (differential pressure transducer; Buxco) and preamplifier (MAX II, Buxco) in the system. The relative increase ratio of Penh was calculated as Penh %=Penh_{methacholine}/Penh_{PBS}, wherein Penh_{methacholine} is the average value of Penh after the mice inhale 3 minutes of methacholine aerosol, and Penh_{PBS} is the average value of Penh after the mice inhale 3 minutes of PBS aerosol.

### (5) Bronchoalveolar Lavage Fluid (BALF) and Lung Histology:

As all groups of mice had finished of AHR analysis, they were sacrificed on next day. The lung was immediately lavaged via the trachea cannula (18 GA, angiocath, B.D.) with 1 ml of Hank's balanced salt solution (HBSS, SH30016.01, Hyclone, USA), which contained 2% fetal bovine serum (FBS) and 2 mM 2Na-EDTA for three times. About 3 ml of BALF were collected. The first collected of BALF was centrifuged at 1500 rpm for 5 min at 4° C. Collected supernatants of BALF were stored at −20° C. for determining the levels of cytokines. The second and third collected BALF were combined and centrifuged under the same conditions as the first collected BALF. The cells obtained by decanting the supernatant was hit slightly to separate them, to which the cells obtained from the first centrifugation were added. The total cell numbers were determined with 0.5 ml CM-10, wherein the density of cells was adjusted to 3×10⁵ cells/ml. Cytospin centrifuging machine (Cytospin 4 Cytocentrifuge, Thermo Shandon, USA) was used to immobilize the cells for the experiments carried out thereafter. 200 μl of cell suspension per mouse was centrifuged at 500 rpm for four minutes, and a slide was prepared, on which the cell suspension was dried in the air. The cells were stained with Liu's stain solution (Liu A and Liu B, Delta, 232, Japan). The cells were observed with a microscope (Olympus, BX41TF, Japan) equipped with an oil immersion lens under 1000 times of magnification. Two hundreds of leukocytes were counted on each slide, which includes four different cells, eosinophil, neutrophil, lymphocyte and monocyte. The experiment results were presented as percentage of specific sub-population of lymphocytes based on the total BALF cells.

### (6) Eotaxin Level Measurement:

The Eotaxin level in bronchoalveolar lavage was measured by Sandwich-ELISA with a kit sold under a trademark of R&D. Briefly, ELISA plates were first covered with a specific Abs and left over night at 4° C. The plates were treated with 1% PBS-BSA and washed before conducting the experiments. Supernatants were then added to the ELISA plates, which were kept at room temperatures for two hours, followed by adding biotin-conjugated Abs. After two more hours at room temperature, avidin-conjugated HRP was then added and left still for two hours. The substrate tetramethylbenzidine (TMB) was then added for coloring, and the wavelength absorbed was measured at OD450 wavelength. The concentrations were calculated by interpolation based on the standard values.

### (7) Biological Statistical Analysis:

All data were presented as mean±SD. All analysis for statistically significant differences was performed with Student's t test as compared with treatment to the As group. P values<0.05 were considered significant.

TABLE 1
Effects of extract and constituents of Poria cocos on airway
hyperresponsiveness (AHR) in mice sensitized with OVA
(Penh %)

Methacholine (mg/mL)

| Group | 25 | 50 |
|---|---|---|
| As | 14.2 ± 8.4 | 18.4 ± 8.5 |
| Pred | 10.4 ± 5.1 | 13.0 ± 5.4 |
| 1PCE | 12.5 ± 5.5 | 17.6 ± 6.6 |
| 2PCE | 13.6 ± 10.2 | 21.1 ± 14.8 |
| 1K1 | 6.9 ± 3.3 | 9.3 ± 4.2* |
| 2K1 | 13.5 ± 5.7 | 20.1 ± 14.4 |
| 1K2 | 12.4 ± 12.0 | 14.7 ± 12.5 |
| 2K2 | 6.1 ± 3.7* | 9.0 ± 5.2* |
| 1K3 | 6.6 ± 6.0 | 8.8 ± 7.7* |
| 2K3 | 9.9 ± 8.2 | 14.9 ± 11.3 |

*p < 0.05

TABLE 2
Effects of extract and constituents of Poria cocos on percentage of
subpopulation of immune cells in BALF of OVA-sensitized mice

| | Monocyte | Lymphocyte | Neutrophil | Eosinophil |
|---|---|---|---|---|
| Group | % | | | |
| As | 19.1 ± 7.9 | 64.7 ± 12.2 | 0.6 ± 0.6 | 15.7 ± 8.3 |
| Pred | 13.3 ± 7.8 | 76.4 ± 9.3* | 0.8 ± 0.8 | 9.4 ± 2.5 |
| 1PCE | 16.5 ± 6.5 | 69.4 ± 12.9 | 0.1 ± 0.2* | 15.1 ± 8.0 |
| 2PCE | 14.2 ± 7.7 | 74.7 ± 12.5 | 0.1 ± 0.2* | 11.0 ± 5.8 |
| 1K1 | 15.1 ± 6.6 | 72.1 ± 10.5 | 0 ± 0* | 12.9 ± 6.3 |
| 2K1 | 18.5 ± 10.7 | 67.6 ± 18.8 | 1.0 ± 0.4* | 13.9 ± 9.0 |
| 1K2 | 31.0 ± 6.6 | 57.9 ± 7.2 | 0.4 ± 0.8 | 10.6 ± 3.2 |
| 2K2 | 35.7 ± 7.5 | 50.4 ± 8.2 | 0.6 ± 0.5 | 13.4 ± 3.8 |
| 1K3 | 38.2 ± 8.7 | 49.8 ± 12.8 | 0.8 ± 0.9 | 11.3 ± 7.0 |
| 2K3 | 37.1 ± 5.7 | 52.1 ± 8.1 | 1.1 ± 1.1 | 9.7 ± 3.6 |

*p < 0.05

TABLE 3
Effects of extract and constituents of Poria cocos on levels
of eotaxin in BALF of OVA-sensitized mice

| Group | Eotaxin (pg/mL) |
|---|---|
| As | 34.1 ± 9.3 |
| Pred | 31.1 ± 6.8 |
| 1PCE | 20.9 ± 6.4* |
| 2PCE | 27.1 ± 11.1 |
| 1K1 | 19.6 ± 8.8* |
| 2K1 | 21.5 ± 5.8* |
| 1K2 | 22.1 ± 13.1 |
| 2K2 | 19.3 ± 6.5* |
| 1K3 | 21.1 ± 10.4* |
| 2K3 | 25.1 ± 12.0 |

*p < 0.05

### Results:

1. It can be seen from Table 1 that, as the concentration of methacholine increases, the value of AHR also increases among groups. The 2K2 group shows the lowest AHR values as compared to the As group, and the 1K1, the 2K2) and the 1K3 groups also have significantly lower values of AHR in comparison with the As group upon higher stimulation of methacholine (50 mg/mL). As the higher values of AHR indicate that the mice suffer from more severity of airway hyperresponsiveness response. The resultant data indicate that the extract and constituents of Poria cocas of the present invention have preventive/therapeutic effects on asthma. The Pred group shows lower values of AHR but not statistically significant to the As group. Therefore, the extract and constituents of Poria cocos of the present invention are more advantageous in preventive/therapeutic effects on asthma in term of the contraction of airway caused by asthma, in comparison with the popular clinical therapeutic steroids for asthma treatment.

2. It can be seen from the results shown in Table 2 that the As group has the highest percentage and the Pred group has the lowest percentage of eosinophil in BALF of OVA-sensitized mice but without statistical significance. However, the percentage of lymphocyte in the Pred group is significantly higher than that of the As group. In spite of that, mice which have been administered with 1PCE, 2PCE, 1K1, and 2K1 show significantly lower percentages of neutrophil in the BALF in comparison with the As group. The results indicate that the constituents of Poria cocos can attenuate the infiltration of inflammatory cells sustained in the lung tissue.

3. On the active phase of asthma initiation stages, there are many kinds of inflammatory proteins involved in the local inflammation. In this example, we analyzed the levels of Eotaxin in the BALF, and the results are shown in Table 3. There are many kinds of cells can secrete Eotaxin, such as the epithelial cell on the airway bronchia, the endothelial cells and the fibroblast cells. Secreted Eotaxin can trigger the eosinophils or Th2 cells to airway bronchia (Rankin et al., Eotaxin and eosinophil recruitment: implication for human disease. Mol Med Today 6:20-27. (2000)). It can be seen from data in Table 3 that mice which have been administered with 1PCE, 1K1, 2K1, and 1K3 show significantly lower levels of Eotaxin in BALF in comparison with the As group. As to the mice in the Pred group show no significant difference on the levels of Eotaxin in comparison with the As group. The results support that the constituents of Poria cocos disclosed in the present invention can prevent the airway from the infiltration of inflammatory cells by inhibiting the secretion of inflammatory protein Eotaxin. Therefore, the constituents of Poria cocos of the present invention are more advantageous in preventive/therapeutic effects on asthma in term of attenuating the infiltration of inflammatory cells (eosinophils or Th2 lymphocytes) sustained in the lung tissue, in comparison with the popular clinical therapeutic steroids (Pred) for asthma treatment.

Although particular embodiments of the invention have been described in details for purposes of illustration, it will be understood by one of ordinary skill in the art that numerous variations will be possible to the disclosed embodiments without going outside the scope of the invention as disclosed in the claims.

1. A method for treating a disease induced from immune disorder in a mammal, which comprises administering to the mammal an amount effective for treating said disease of a lanostane having the following chemical formula (I) or a pharmaceutically acceptable salt thereof: 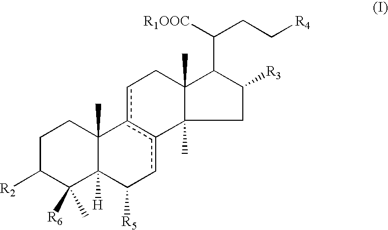
wherein R₁ is either H or CH₃; R₂ is OCOCH₃, ═O or OH; R₃ is H or OH; R₄ is —C(═CH₂)—C(CH₃)₂Rₐ, in which Rₐ is H or OH, or —CH═C(CH₃)—R_{b}, in which R_{b} is CH₃ or CH₂OH; R₅ is H or OH; and R₆ is CH₃ or CH₂OH.

2. The method as defined in claim 1, wherein said disease induced from immune disorder is an allergy.

3. The method as defined in claim 2, wherein said allergy is allergic rhinitis, allergic conjunctivitis, allergic asthma, atopic dermatitis, food allergy, atopic eczema, or rheumatoid arthritis.

4. The method as defined in claim 3, wherein said allergy is allergic asthma.

5. The method as defined in claim 1, wherein the lanostane (I) is

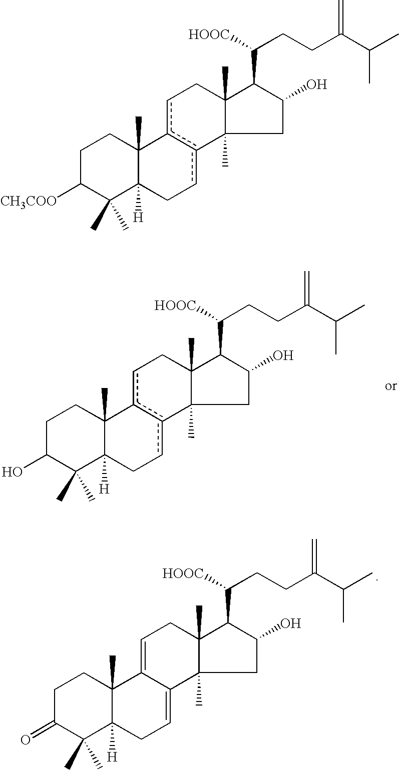

6. The method as defined in claim 1, which comprises administering to the mammal a pharmaceutical composition containing 0.1-60 wt % of the lanostane (I) or a pharmaceutically acceptable salt thereof.

7. The method as defined in claim 3, wherein the pharmaceutical composition is administered to the mammal orally.

8. The method as defined in claim 1, wherein the mammal is a human.

9. The method as defined in claim 1, which comprises administering to the mammal a Poria extract comprising 1-60% of the lanostane (I) by weight of the extract, and being substantially devoid of secolanostane.

10. The method as defined in claim 9, wherein said Poria extract is prepared by a method comprising the following steps:
a) extracting metabolites, fermentation products or sclerotium of Poria cocos (Schw) Wolf by water, methanol, ethanol, or a mixed solvent thereof;
b) concentrating the resulting liquid extract from step a);
c) introducing the resulting concentrated substance from step b) into a silica gel column;
d) eluting the silica gel column with an eluent having a low polarity, and collecting the resulting eluate; and
e) concentrating the eluate to form a concentrated eluate.

11. The method as defined in claim 10, wherein the concentrated eluate from step e) has a chromatographic value, Rf, not less than 0.1 in accordance with a thin layer chromatography, which is developed by a mixed solvent of dichloromethane:methanol=96:4 and is detected by an ultraviolet lamp and iodine vapor.

12. The method as defined in claim 10, wherein the extraction in step a) is carried out by using 95% ethanol.

13. The method as defined in claim 10, wherein the extraction in step a) comprises extracting metabolites, fermentation products or sclerotium of Poria cocos (Schw) Wolf by boiling water; adding a base to the resulting extraction aqueous solution until a pH value thereof is 9-11; recovering the basic aqueous solution; adding an acid to the basic aqueous solution until a pH value thereof is 4-6 to form a precipitate; recovering the precipitate; extracting the precipitate with ethanol; and recovering a liquid extract.

14. The method as defined in claim 12, wherein the concentrated substance resulted from step b) is further extracted with a two-phase solvent containing methanol and n-hexane in a volumetric ratio of 1:1, a methanol layer is separated from the two-phase solvent extraction mixture, and the methanol layer is concentrated to form a concentrate, which is used as a feed to the silica gel column in step c).

15. The method as defined in claim 13, wherein the concentrated substance resulted from step b) is further extracted with a two-phase solvent containing methanol and n-hexane in a volumetric ratio of 1:1, a methanol layer is separated from the two-phase solvent extraction mixture, and the methanol layer is concentrated to form a concentrate, which is used as a feed to the silica gel column in step c).

16. The method as defined in claim 10, wherein the low polarity eluent in step
d) is a mixed solvent containing dichloromethane and methanol in a volumetric ratio of 96.5:3.5.

17. The method as defined in claim 9, wherein said Poria extract comprises 5-35% of the lanostane (I).

18. The method as defined in claim 9, wherein the lanostane (I) is [chemical structure]
